(19) United States
(12) Patent Application Publication
(10) Pub. No.: US 2009/0270416 A1
(43) Pub. Date: Oct. 29, 2009

(54) N-OXIDES OF DIARYLUREA DERIVATIVES AND THEIR USE AS Chk1 INHIBITORS FOR THE TREATMENT OF CANCER

(75) Inventors: Robert George Boyle, Cambridge (GB); Stuart Travers, Shefford (GB)

Correspondence Address: HESLIN ROTHENBERG FARLEY & MESITI PC, 5 COLUMBIA CIRCLE, ALBANY, NY, 12203, US

(73) Assignee: SENTINEL ONCOLOGY LIMITED, Cambridge (GB)

(21) Appl. No.: 12/304,345

(22) PCT Filed: Jun. 8, 2007
(86) PCT No.: PCT/GB2007/002123

§ 371 Date: Dec. 11, 2008

Related U.S. Application Data
(60) Provisional application No. 60/804,488, filed on Jun. 12, 2006.

(30) Foreign Application Priority Data
Jun. 12, 2006 (GB) 0611565.3

(51) Int. Cl. A61K 31/4965; C07D 241/02; C07D 401/04

(52) U.S. Cl. 514/255.05; 544/336; 514/255.06; 544/405

(57) ABSTRACT

The invention provides a Chk-1 kinase inhibiting compound of the formula (I) or a salt, solvate or tautomer thereof, wherein: G is CH₂, O, NH, NHCO or CONH; A is a group (CH₂)ₙ where n is 1 to 4 provided that when G is O or NH, n is at least 2; X¹ is nitrogen or CH; X² is nitrogen or a group CR⁵; X³ is nitrogen or a group CR⁵; X⁴ is nitrogen or CH; provided that no more than two of X², X³ and X⁴ are nitrogen; and R¹; R²; R³; R⁴; R⁵ and R⁶ are as defined in the claims.

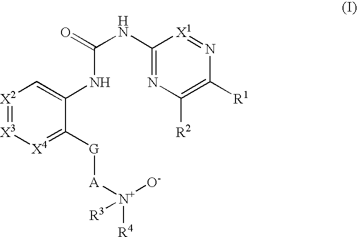

This invention relates to compounds that inhibit or modulate the activity of Chk-1 kinase. Also provided are pharmaceutical compositions containing the compounds and the therapeutic uses of the compounds.

### BACKGROUND OF THE INVENTION

Chk-1 is a serine/threonine kinase involved in the induction of cell cycle checkpoints in response to DNA damage and replicative stress. Cell cycle checkpoints are regulatory pathways that control the order and timing of cell cycle transitions. They ensure that critical events such as DNA replication and chromosome segregation function correctly. The regulation of these cell cycle checkpoints is of considerable importance in determining the manner in which tumour cells respond to chemotherapy and radiation therapy. Many anti-cancer drugs achieve their anti-cancer effects by causing DNA damage but resistance to such drugs is a significant problem. One mechanism responsible for drug resistance is attributed to the prevention of cell cycle progression through the control of critical activation of a checkpoint pathway. This arrests the cell cycle to provide time for repair, and induces the transcription of genes to facilitate repair, thereby avoiding immediate cell death. By preventing checkpoint arrests at, for example, the G2 checkpoint, it should provide possible to increase the extent of tumour cell death induced by DNA damage and circumvent resistance. Human Chk-1 plays a role in regulating cell cycle arrest by phosphorylating the phosphatase cdc25 on Serine 216, which may be involved in preventing activation of cdc2/cyclin B and initiating mitosis. Therefore, it is envisaged that inhibition of Chk-1 should enhance DNA damaging agents by initiating mitosis before DNA repair is complete and thereby causing tumour cell death.

It is also envisaged that Chk1 inhibitors may be useful in treating tumour cells in which the G1/S DNA damage checkpoint has been lost and where the tumours therefore rely on the G2/M DNA damage checkpoint exclusively to correct any DNA damage. Examples of such tumours are those arising from or associated with mutations in the p53 gene, a tumour suppressor gene found in about 50% of all human cancers (see for example Hahn et al., “Rules for making human tumor cells” N Engl J Med 2002; 347: 1593-603 and Hollstein et al., “p53 mutations in human cancers. Science 1991; 253: 49-53). Thus, Chk1 inhibitors should be of particular value in treating p53 negative or mutated tumours.

Various attempts have been made to develop inhibitors of Chk-1 kinase. For example, WO 03/10444 and WO 2005/072733 (both in the name of Millennium) disclose aryl/heteroaryl urea compounds as Chk-1 kinase inhibitors. US2005/215556 (Abbott) discloses macrocyclic ureas as kinase inhibitors. WO 02/070494, WO2006014359 and WO2006021002 (all in the name of Icos) disclose aryl and heteroaryl ureas as Chk-1 inhibitors.

Recently, a great deal of attention has been given to the ion-channel blocking activities of drug candidates and in particular the ability of drug candidates to block the HERG ion channel. In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

The separation of HERG blocking activity and therapeutically useful effects such as kinase inhibition is currently considered to be of substantial importance in the development of any new drug. Typically there should be at least a tenfold difference between the level of activity against the therapeutic target and the level of hERG blocking activity for a particular compound to be considered worthy of further development as a drug candidate.

### SUMMARY OF THE INVENTION

Many of the compounds disclosed in WO 03/10444 contain a 2-aminoalkoxyphenyl group or substituted aminoalkoxyphenyl group attached to one of the nitrogen atoms of a urea moiety. It has now been found that by forming an N-oxide with the amino group of the 2-aminoalkoxyphenyl group, the hERG activity of the compound is dramatically reduced whilst potency against the Chk-1 kinase is maintained.

Accordingly, in a first aspect, the invention provides a compound of the formula (I):

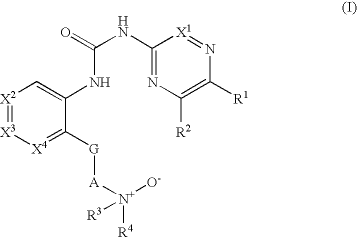

or a salt, solvate or tautomer thereof, wherein:
G is CH₂, O, NH, NHCO or CONH;
A is a group (CH₂)ₙ where n is 1 to 4 provided that when G is O or NH, n is at least 2;
X¹ is nitrogen or CH;
X² is nitrogen or a group CR⁵;
X³ is nitrogen or a group CR⁵;
X⁴ is nitrogen or CH; provided that no more than two of X², X³ and X⁴ are nitrogen;
R¹ is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups;
R² is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups; provided that no more than one of R¹ and R² can be an aryl or heteroaryl group;
or R¹ and R² together with the carbon atoms to which they are attached form a benzene ring;
R³ and R⁴ are the same or different and each is C₁₋₄ alkyl; or R³ and R⁴ together with the nitrogen atom to which they are attached form an azetidine, pyrrolidine, piperidine, piperazine, M-methylpiperazine or morpholine group; or R³ together with the nitrogen atom to which it is attached and the moiety A together form a saturated 5 to 7 membered heterocyclic ring optionally containing a second heteroatom ring member selected from O and S, wherein the heterocyclic ring is optionally substituted by 1 to 4 methyl groups, and R⁴ is C₁₋₄ alkyl;
R⁵ is hydrogen or a substituent R⁶;
R⁶ is halogen; hydroxy; trifluoromethyl; cyano; nitro; amino; mono- or di-C₁₋₄ hydrocarbylamino; a carbocyclic or heterocyclic group having from 3 to 12 ring members and optionally substituted by one or more substituents R⁷; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is:
hydrogen;
a carbocyclic and heterocyclic group having from 3 to 12 ring members and being optionally substituted by one or more substituents R⁷;
a C₁₋₁₂ hydrocarbyl group optionally substituted by one or more substituents selected from hydroxy; oxo; halogen; cyano; nitro; carboxy; amino; mono- or di-C₁₋₈ non-aromatic hydrocarbylamino; and carbocyclic and heterocyclic groups having from 3 to 12 ring members optionally substituted by one or more substituents R⁷; wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c} is R^{b}, hydrogen or C₁₋₄ hydrocarbyl;
X¹ is O, S or NR^{c}; and
X² is ═O, ═S or ═NR^{c};
wherein R⁷ is selected from R⁶ provided that when the substituents R⁷ contain a carbocyclic or heterocyclic group having from 3 to 12 ring members, the said carbocyclic or heterocyclic group can be unsubstituted or substituted by one or more substituents R⁸; and
R⁸ is selected from R⁶ except that any carbocyclic or heterocyclic groups constituting or forming part of R⁸ may not bear a substituent containing or consisting of a carbocyclic or heterocyclic group but may optionally bear one or more substituents selected from halogen; hydroxy; trifluoromethyl; cyano; nitro; amino; mono- or di-C₁₋₄ hydrocarbylamino; or a group R^{a}—R^{bb}; where R^{a} is as hereinbefore defined and R^{bb} is hydrogen or a C₁₋₆ hydrocarbyl group optionally substituted by one or more substituents selected from hydroxy, oxo, halogen, cyano, nitro, carboxy, amino, mono- or di-C₁₋₄ saturated hydrocarbylamino and wherein one or more carbon atoms of the C₁₋₆ hydrocarbyl group may optionally be replaced by O, S, SO, SO₂, NR^{c}, X¹C(X²), C(X²)X¹ or X¹C(X²)X¹.

In another aspect, the invention provides a compound of the formula (Ia):

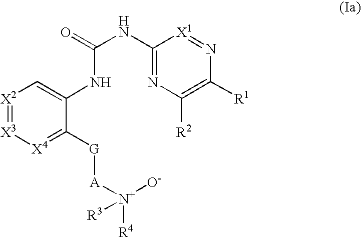

or a salt, solvate or tautomer thereof, wherein:
G is CH₂, O, NH, NHCO or CONH;
A is a group (CH₂)ₙ where n is 1 to 4 provided that when G is O or NH, n is at least 2;
X¹ is nitrogen or CH;
X² is nitrogen or a group CR⁵;
X³ is nitrogen or a group CR⁵;
X⁴ is nitrogen or CH; provided that no more than two of X², X³ and X⁴ are nitrogen;
R¹ is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups;
R² is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups; provided that no more than one of R¹ and R² can be an aryl or heteroaryl group;
or R¹ and R² together with the carbon atoms to which they are attached form a benzene ring;
R³ and R⁴ are the same or different and each is C₁₋₄ alkyl; or R³ and R⁴ together with the nitrogen atom to which they are attached form an azetidine, pyrrolidine, piperidine, piperazine, M-methylpiperazine or morpholine group; and
R⁵ is hydrogen or a substituent R⁶;
R⁶ is halogen; hydroxy; trifluoromethyl; cyano; nitro; amino; mono- or di-C₁₋₄ hydrocarbylamino; a carbocyclic or heterocyclic group having from 3 to 12 ring members and optionally substituted by one or more substituents R⁷; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is:
hydrogen;
a carbocyclic and heterocyclic group having from 3 to 12 ring members and being optionally substituted by one or more substituents R⁷;
a C₁₋₁₂ hydrocarbyl group optionally substituted by one or more substituents selected from hydroxy; oxo; halogen; cyano; nitro; carboxy; amino; mono- or di-C₁₋₈ non-aromatic hydrocarbylamino; and carbocyclic and heterocyclic groups having from 3 to 12 ring members optionally substituted by one or more substituents R⁷; wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c}C is R^{b}, hydrogen or C₁₋₄ hydrocarbyl;
X¹ is O, S or NR^{c}; and
X² is ═O, ═S or ═NR^{c};
wherein R⁷ is selected from R⁶ provided that when the substituents R⁷ contain a carbocyclic or heterocyclic group having from 3 to 12 ring members, the said carbocyclic or heterocyclic group can be unsubstituted or substituted by one or more substituents R⁸; and
R⁸ is selected from R⁶ except that any carbocyclic or heterocyclic groups constituting or forming part of R⁸ may not bear a substituent containing or consisting of a carbocyclic or heterocyclic group but may optionally bear one or more substituents selected from halogen; hydroxy; trifluoromethyl; cyano; nitro; amino; mono- or di-C₁₋₄ hydrocarbylamino; or a group R^{a}—R^{bb}; where R^{a} is as hereinbefore defined and R^{bb} is hydrogen or a C₁₋₆ hydrocarbyl group optionally substituted by one or more substituents selected from hydroxy, oxo, halogen, cyano, nitro, carboxy, amino, mono- or di-C₁₋₄ saturated hydrocarbylamino and wherein one or more carbon atoms of the C₁₋₆ hydrocarbyl group may optionally be replaced by O, S, SO, SO₂, NR^{c}, X¹C(X²), C(X²)X¹ or X¹C(X²)X¹.

The invention also provides inter alia:
A compound of the formula (I) as defined herein for use as a Chk1 kinase inhibitor.
A compound of the formula (I) as defined herein for use in enhancing a therapeutic effect of radiation therapy or chemotherapy in the treatment of a proliferative disease such as cancer.
The use of a compound of the formula (I) for the manufacture of a medicament for enhancing a therapeutic effect of radiation therapy or chemotherapy in the treatment of a proliferative disease such as cancer.
A method for the prophylaxis or treatment of a proliferative disease such as cancer, which method comprises administering to a patient in combination with radiotherapy or chemotherapy a compound of the formula (I) as defined herein.
A compound of the formula (I) as defined herein for use in the treatment of a patient suffering from a p53 negative or mutated tumour.
A compound of the formula (I) as defined herein for the use in the treatment of a patient suffering from a p53 negative or mutated tumour in combination with radiotherapy or chemotherapy
The use of a compound of the formula (I) as defined herein for the manufacture of a medicament for the treatment of a patient suffering from a p53 negative or mutated tumour.
A method for the treatment of a patient (e.g. a human patient) suffering from a p53 negative or mutated tumour, which method comprises administering to the patient a therapeutically effective amount of a compound of the formula (I).
A pharmaceutical composition comprising a compound of the formula (I) as defined herein and a pharmaceutically acceptable carrier.
A compound of the formula (I) as defined herein for use in medicine.

### GENERAL PREFERENCES AND DEFINITIONS

In this section, as in all other sections of this application, unless the context indicates otherwise, references to a compound of formula (I) includes all subgroups thereof as defined herein (for example formulae (Ia), (II) and (II) and sub-groups thereof), and the term ‘subgroups’ includes all preferences, embodiments, examples and particular compounds defined herein.

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Examples of bicyclic heteroaryl groups containing a six membered ring fused to a five membered ring include but are not limited to benzofuran, benzothiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzothiene, dihydrobenzofuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine). Other examples of non-aromatic heterocyclic groups include cyclic amide moieties (e.g. as in pyrrolidone) and cyclic ester moieties (e.g. as in butyrolactone).

Examples of monocyclic non-aromatic heterocyclic groups include 5-, 6- and 7-membered monocyclic heterocyclic groups. Particular examples include morpholine, thiomorpholine and its S-oxide and S,S-dioxide, piperidine (e.g. 1-piperidinyl, 2-piperidinyl, 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups. Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

Where present and where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}—R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c} C(O), OC(S), SC(S), NR^{c} C(S), OC(NR^{c}), SC(NR^{c}), NR^{c} C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c} C(O)O, OC(S)O, SC(S)O, NR^{c} C(S)O, OC(NR^{c})O, SC(NR¹)O, NR^{c} C(NR^{c})O, OC(O)S, SC(O)S, NR^{c} C(O)S, OC(S)S, SC(S)S, NR^{c} C(S)S, OC(NR^{c})S, SC(NR^{c})S, NR^{c} C(NR^{c})S, OC(O)NR^{c}, SC(O)NR^{c}, NR^{c} C(O)NR^{c}, OC(S)NR^{c}, SC(S)NR^{c}, NR^{c} C(S)NR^{c}, OC(NR^{c})NR^{c}, SC(NR^{c})NR^{c}, NR^{c} C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkylalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by, for example, a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}—R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl, phenethyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}—R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}—R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}—R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### SPECIFIC EMBODIMENTS AND PREFERENCES

### X¹

X¹ can be nitrogen or CH.

In one embodiment, X¹ is CH.

In another embodiment, X¹ is nitrogen.

### R¹

R¹ is hydrogen or a substituent selected from cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups.

When R¹ is C₁₋₄ alkyl, examples of such groups are methyl, ethyl, n-propyl, isopropyl, n-butyl, isobutyl and tert-butyl groups. More particularly the group is a C₁₋₃ alkyl group or a C₁₋₂ alkyl group. In particular, the C₁₋₄ alkyl group can be a methyl group.

When R¹ is a 5-6 membered monocyclic aryl or heteroaryl group, example of such groups include any of the groups 5-6 membered monocyclic aryl and heteroaryl groups set out above in the General Preferences and Definitions section of this application.

Particular examples of such groups are six membered rings containing one or two nitrogen ring members (preferably one nitrogen ring member) and five membered rings containing a nitrogen ring member and optionally one or two further heteroatom ring members selected from nitrogen, oxygen and sulphur. For example, the 5-6 membered heteroaryl group can be an oxazole, thiazole, isoxazole, isothiazole, imidazole, pyrazole or triazole group.

The aryl and heteroaryl groups can be unsubstituted or substituted by one or two C₁₋₄ alkyl groups, more preferably one or two methyl groups, for example a single methyl group.

One sub-group of moieties for R¹ consists of hydrogen, methyl, trifluoromethyl, cyano, pyridyl, oxazolyl and methyl-substituted triazolyl.

In one particular embodiment, R¹ is hydrogen.

### R²

R² is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups; provided that no more than one of R¹ and R² can be an aryl or heteroaryl group.

When R² is C₁₋₄ alkyl, examples of such groups are methyl, ethyl, n-propyl, isopropyl, n-butyl, isobutyl and tert-butyl groups. More particularly the group is a C₁₋₃ alkyl group or a C₁₋₂ alkyl group. In particular, the C₁₋₄ alkyl group can be a methyl group.

When R² is a 5-6 membered monocyclic aryl or heteroaryl group, example of such groups include any of the groups 5-6 membered monocyclic aryl and heteroaryl groups set out above in the General Preferences and Definitions section of this application.

Particular examples of such groups are six membered rings containing one or two nitrogen ring members (preferably one nitrogen ring member) and five membered rings containing a nitrogen ring member and optionally one or two further heteroatom ring members selected from nitrogen, oxygen and sulphur. For example, the 5-6 membered heteroaryl group can be an oxazole, thiazole, isoxazole, isothiazole, imidazole, pyrazole or triazole group.

The aryl and heteroaryl groups can be unsubstituted or substituted by one or two C₁₋₄ alkyl groups, more preferably one or two methyl groups, for example a single methyl group.

One sub-group of moieties for R² consists of hydrogen, methyl, trifluoromethyl, cyano, pyridyl, oxazolyl and methyl-substituted triazolyl.

In one particular embodiment, R² is hydrogen.

Alternatively, in another embodiment, R¹ and R² together with the carbon atoms to which they are attached form a benzene ring;

Specific examples of the moiety:

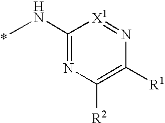

wherein the asterisk indicates the attachment of the moiety to the carbonyl group of the urea, are set out in Table 1 below.

TABLE 1

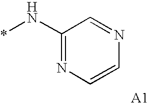

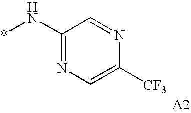

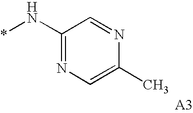

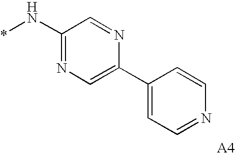

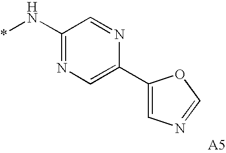

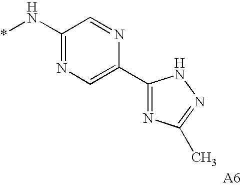

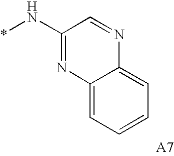

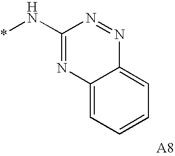

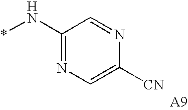

### G

The moiety G is CH₂, O, NH, NHCO or CONH.

More typically, G is CH₂, O or NH.

In one preferred group of compounds, G is O.

### A

A is a group (CH₂)ₙ where n is 1 to 4 provided that when G is O or NH, n is at least 2.

More typically, n is 2 or 3. In one particular group of compounds, n is 3 and hence the group (CH₂)ₙ is a propylene group.

### R³ and R⁴

In the moiety N(O)R³R⁴, R³ and R⁴ are the same or different and each is C₁₋₄ alkyl; or R³ and R⁴ together with the nitrogen atom to which they are attached form an azetidine, pyrrolidine, piperidine, piperazine, N-methylpiperazine or morpholine group; or R³ together with the nitrogen atom to which it is attached and the moiety A together form a saturated 5 to 7 membered heterocyclic ring optionally containing a second heteroatom ring member selected from O and S, wherein the heterocyclic ring is optionally substituted by 1 to 4 methyl groups, and R⁴ is C₁₋₄ alkyl.

In one embodiment, R³ and R⁴ are the same or different and each is C₁₋₄ alkyl; or R³ and R⁴ together with the nitrogen atom to which they are attached form an azetidine, pyrrolidine, piperidine, piperazine, N-methylpiperazine or morpholine group.

When R³ and R⁴ are C₁₋₄ alkyl, preferred alkyl groups are C₁₋₃ alkyl group and C₁₋₂ alkyl groups. Examples of combinations of R³ and R⁴ groups are (i) R³=methyl, R⁴ methyl; (ii) R³=methyl, R⁴=ethyl; (iii) R³=ethyl, R⁴=ethyl; (iv) R³=methyl, R⁴=n-propyl; (v) R³=methyl, R⁴=isopropyl; (vi) R³=ethyl, R⁴=n-propyl; and (vii) R³=ethyl, R⁴=isopropyl.

One particular combination of R³ and R⁴ groups is (i) R³=methyl, R⁴=methyl.

In another embodiment, R³ together with the nitrogen atom to which it is attached and the moiety A together form a saturated 5 to 7 membered heterocyclic ring optionally containing a second heteroatom ring member selected from O and S, wherein the heterocyclic ring is optionally substituted by 1 to 4 methyl groups.

In this embodiment, particular heterocyclic rings are pyrrolidine, piperidine, azepine, morpholine and thiomorpholine rings, and preferred heterocyclic rings are pyrrolidine, piperidine and morpholine rings.

The heterocyclic ring may be unsubstituted or substituted by 1 to 4 methyl groups. Typically the heterocyclic ring will be substituted by 0-3, more typically 0-2 methyl groups, for example 0 or 1 methyl groups. In one embodiment, the heterocyclic ring is unsubstituted.

In this embodiment, most preferably, R³ together with the nitrogen atom to which it is attached and the moiety A together form an unsubstituted piperidine ring.

Also in this embodiment, R⁴ is preferably a methyl group.

### X² and X³

Each of X² and X³ can be nitrogen or a group CR⁵, provided that no more than two of X², X³ and X⁴ are nitrogen.

In one particular embodiment, X² is CH or a group CR^{6a}, where R^{6a} is:
halogen;
hydroxy;
cyano;
nitro;
amino; mono- or di-C₁₋₄ alkylamino;
C₁₋₄ alkyl optionally substituted by one or more fluorine atoms, hydroxy, C₁₋₂ alkoxy, cyano, amino or mono- or di-C₁₋₄ alkylamino;
C₁₋₄ alkoxy optionally substituted by one or more fluorine atoms or C₁₋₂ alkoxy;
2-hydroxyethoxy; or
2-aminoethoxy;
   and X³ is CH or a group CR⁶ as hereinbefore defined.

Within this embodiment, examples of X² are CH and CR^{6a} where R^{6a} is:
fluorine;
chlorine;
hydroxy;
cyano;
amino or mono- or di-C₁₋₂ alkylamino;
C₁₋₃ alkyl optionally substituted by one or more fluorine atoms, hydroxy, C₁₋₂ alkoxy, cyano or amino or mono- or di-C₁₋₂ alkylamino;
C₁₋₂ alkoxy optionally substituted by one or more fluorine atoms or C₁₋₂ alkoxy;
2-hydroxyethoxy; or
2-aminoethoxy.

More particularly, X² is CH or C—Cl.

Examples of X³ are CH and CR^{6b} where R^{6b} is halogen; hydroxy; trifluoromethyl; cyano; amino; mono- or di-C₁₋₄ hydrocarbylamino; a carbocyclic group of 3 to 6 ring members or a heterocyclic group of 5 to 6 ring members, the carbocyclic and heterocyclic groups being optionally substituted by one or more substituents R^{7a}; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is:
hydrogen;
a carbocyclic group of 3 to 6 ring members or a heterocyclic group of 5 to 6 ring members being optionally substituted by one or more substituents R^{7a};
a non-aromatic C₁₋₁₂ hydrocarbyl group optionally substituted by one or more substituents selected from hydroxy, oxo, halogen, cyano, carboxy, amino, mono- or di-C₁₋₈ non-aromatic hydrocarbylamino, a carbocyclic group of 3 to 6 ring members or a heterocyclic group of 5 to 6 ring members, the carbocyclic and heterocyclic groups being optionally substituted by one or more substituents R^{7a}; and wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c} is R^{b}, hydrogen or C₁₋₄ hydrocarbyl;
X¹ is O, S or NR^{c}; and
X² is ═O, ═S or ═NR^{c};
wherein R^{7a} is the same as R^{6b} provided that when the substituents R^{7a} contain a carbocyclic or heterocyclic group having from 3 to 12 ring members, the said carbocyclic or heterocyclic group can be unsubstituted or substituted by one or more substituents R^{8a}; and
R^{8a} is the same as R^{6b} except that any carbocyclic or heterocyclic groups constituting or forming part of R^{8a} may not bear a substituent containing or consisting of a carbocyclic or heterocyclic group but may optionally bear one or more substituents selected from
halogen;
hydroxy;
cyano;
nitro;
amino; mono- or di-C₁₋₄ alkylamino;
C₁₋₄ alkyl optionally substituted by one or more fluorine atoms, hydroxy, C₁₋₂ alkoxy, cyano, amino or mono- or di-C₁₋₄ alkylamino;
C₁₋₄ alkoxy optionally substituted by one or more fluorine atoms or C₁₋₂ alkoxy;
2-hydroxyethoxy; or
2-aminoethoxy.

Particular examples of the moiety:

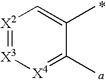

wherein the asterisk * denotes the point of attachment to the urea group and “a” denotes the point of attachment to the group G, are shown in Table 2 below.

TABLE 2

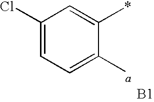

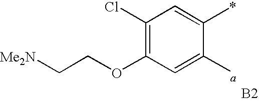

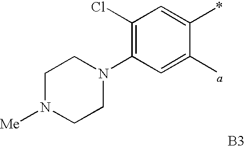

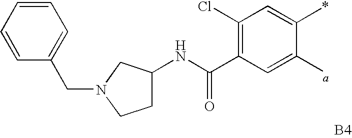

One preferred group is group B1.

### Preferred Sub-Groups of Compounds

One preferred sub-group of compounds can be represented by the general formula (II)

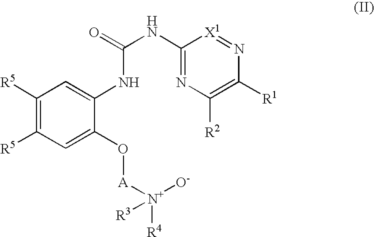

or a salt, solvate or tautomer thereof;
wherein R¹, R², R³, R⁴, R⁵, A and X¹ are as hereinbefore defined.

Within formula (II), one group of compounds can be represented by the formula (III):

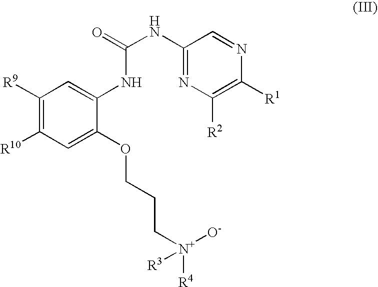

or a salt, solvate or tautomer thereof;
wherein R¹, R², R³ and R⁴ are as hereinbefore defined;
R⁹ is hydrogen or a substituent selected from:
halogen;
hydroxy;
cyano;
amino; mono- or di-C₁₋₂ alkylamino;
C₁₋₄ alkyl optionally substituted by one or more fluorine atoms, hydroxy, C₁₋₂ alkoxy, cyano, amino or mono- or di-C₁₋₄ alkylamino;
C₁₋₄ alkoxy optionally substituted by one or more fluorine atoms or C₁₋₂ alkoxy;
2-hydroxyethoxy; or
2-aminoethoxy; and
   R¹⁰ is hydrogen or a substituent selected from halogen; hydroxy; trifluoromethyl; cyano; amino; mono- or di-C₁₋₄ hydrocarbylamino; a carbocyclic group of 3 to 6 ring members or a heterocyclic group of 5 to 6 ring members, the carbocyclic and heterocyclic groups being optionally substituted by one or more substituents R^{7a}; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c} SO₂;
R^{b} is:
hydrogen;
a carbocyclic group of 3 to 6 ring members or a heterocyclic group of 5 to 6 ring members being optionally substituted by one or more substituents R^{7a};
a non-aromatic C₁₋₁₂ hydrocarbyl group optionally substituted by one or more substituents selected from hydroxy, oxo, halogen, cyano, carboxy, amino, mono- or di-C₁₋₈ non-aromatic hydrocarbylamino, a carbocyclic group of 3 to 6 ring members or a heterocyclic group of 5 to 6 ring members, the carbocyclic and heterocyclic groups being optionally substituted by one or more substituents R^{7a}; and wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c} is R^{b}, hydrogen or C₁₋₄ hydrocarbyl;
X¹ is O, S or NR^{c}; and
X² is ═O, ═S or ═NR^{c}
   wherein R^{7a} is as hereinbefore defined.

Particular groups of compounds within formula (III) are the compounds wherein the moiety:

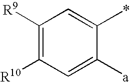

is a group as set out in Table 2 above.

For the avoidance of doubt, it is to be understood that each general and specific preference, embodiment and example of one R group may be combined with each general and specific preference, embodiment and example of each other R group, X¹, X², X³, X⁴, G and A as defined herein and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular and preferred compounds are as set out in the examples.

### Salts, Solvates, Tautomers, Isomers, Prodrugs and Isotopes

A reference to a compound of the formulae (I) and sub-groups thereof also includes ionic forms, salts, solvates, isomers, tautomers, prodrugs, isotopes and protected forms thereof, for example, as discussed below.

Many compounds of the formula (I) can exist in the form of salts, for example acid addition salts or, in certain cases salts of organic and inorganic bases such as phenolate, carboxylate, sulphonate and phosphate salts. All such salts are within the scope of this invention, and references to compounds of the formula (I) include the salt forms of the compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

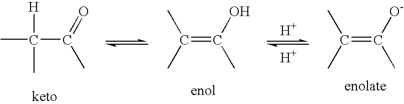

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇ alkyl
(e.g., -Me, -Et, -nPr, -iPr, -nBu, -sBu, -iBu, -tBu);
C₁₋₇ aminoalkyl
(e.g., aminoethyl; 2-(N,N-diethylamino)ethyl; 2-(4-morpholino)ethyl); and
acyloxy-C₁₋₇ alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy)carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Biological Activity

The compounds of the formulae (I) and sub-groups thereof are inhibitors of Chk1 and consequently are expected to be beneficial in combination with various chemotherapeutic agents or radiation for treating a wide spectrum of proliferative disorders.

Examples of such proliferative disorders include, but are not limited to carcinomas, for example carcinomas of the bladder, breast, colon, kidney, epidermis, liver, lung, esophagus, gall bladder, ovary, pancreas, stomach, cervix, thyroid, prostate, gastrointestinal system, or skin, hematopoietic tumours such as leukaemia, B-cell lymphoma, T-cell lymphoma, Hodgkin's lymphoma, non-Hodgkin's lymphoma, hairy cell lymphoma, or Burkett's lymphoma; hematopoietic tumours of myeloid lineage, for example acute and chronic myelogenous leukaemias, myelodysplastic syndrome, or promyelocytic leukaemia; thyroid follicular cancer; tumours of mesenchymal origin, for example fibrosarcoma or rhabdomyosarcoma; tumours of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The Chk-1 inhibitor compounds of the invention may be used in combination with DNA-damaging anti-cancer drugs and/or radiation therapy to treat subjects with multi-drug resistant cancers. A cancer is considered to be resistant to a drug when it resumes a normal rate of tumour growth while undergoing treatment with the drug after the tumour had initially responded to the drug. A tumour is considered to “responds to a drug” when it exhibits a decrease in tumor mass or a decrease in the rate of tumour growth.

It is also envisaged that the Chk1 inhibitors of the invention may be useful in treating tumours in which mutations (e.g. in p53) have led to the G1/S DNA damage checkpoint being lost (see the introductory section of this application)

### Methods for the Preparation of Compounds of the Formula (I)

In this section, as in all other sections of this application unless the context indicates otherwise, references to Formula (I) also include all sub-groups and examples thereof as

Compounds of the formula (I) can be prepared in accordance with synthetic methods well known to the skilled person.

For example, the compounds of formula (I) can be prepared by the reaction of a compound of formula (X):

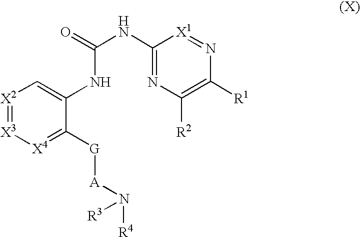

wherein X¹, X², X³, X⁴, G, A, R¹, R², R³, and R⁴ are as hereinbefore defined, with a reagent capable of selectively oxidizing a non-aromatic amine to an N-oxide in the presence of a basic heteroaromatic nitrogen atom.

Examples of reagents capable of oxidizing a non-aromatic amine to an N-oxide in the presence of a basic heteroaromatic nitrogen atom are arylsulphonyloxaziridines such as 2-benzenesulphonyl-3-phenyl-oxaziridine which has the structure (XI):

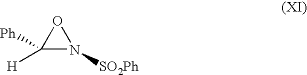

2-Benzenesulphonyl-3-phenyl-oxaziridine can be prepared by the methods set out in the examples.

Compounds of the formula (X) can be prepared by the reaction of a compound of the formula (XI):

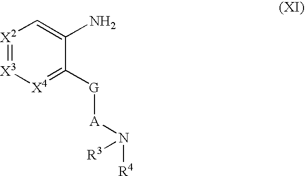

with a carbonyl azide of the formula (XII):

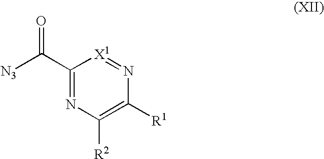

Azides of the formula (XII) can be prepared from the corresponding carboxylic acid of the formula (XIII):

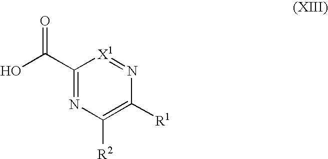

by reaction with diphenylphosphorylazide in a polar non-protic solvent such as tetrahydrofuran (THF) in the presence of a non-interfering base such as triethylamine. The reaction is typically carried out at room temperature.

Alternatively, the azide can be made by forming an acid chloride of the carboxylic acid, and reacting the acid chloride with sodium azide.

Compounds of the formulae (XIII) can be obtained commercially or can be made using standard methods well known to the skilled chemist.

Compounds of the formula (XI) wherein G is O and X⁴ is CH can be prepared by the reaction of a compound of the formula (XIV):

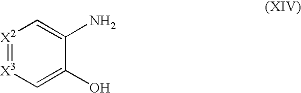

with a compound of the formula L-A-NR³R⁴, wherein L is a leaving group such as a halogen, e.g. chlorine. The reaction is typically carried out in a polar solvent or a mixture of a polar solvent (e.g. isopropanol) and a non-polar solvent (e.g. an aromatic hydrocarbon such as toluene), in the presence of a strong base such as an alkaline metal alkoxide (e.g. sodium methoxide) in order to generate a phenolate anion. The reaction mixture may be heated, e.g. to the reflux temperature of the solvent.

Compounds of the formula (XIV) are commercially available or can be made using standard methods well known to the skilled person.

Compounds of the formula (XI) wherein G is NHCO can be prepared by the sequence of reactions shown in Scheme 1.

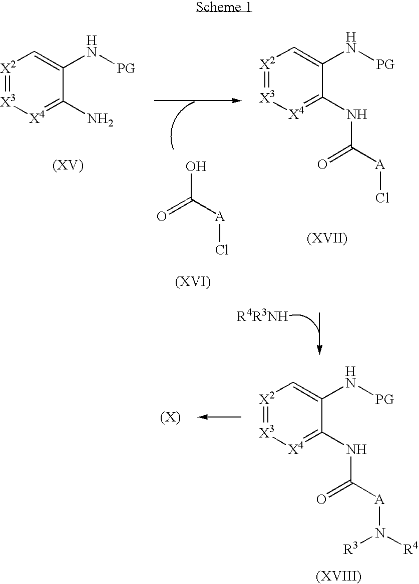

In Scheme 1, a mono-protected amine (XV), where PG is a protecting group such as tert-butyloxycarbonyl (boc), is coupled with the carboxylic acid (XVI), or an activated derivative thereof, under amide forming conditions to give the chloro-amide (XVII).

The coupling reaction between the carboxylic acid (XVI) and the amine (XV) is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The chloro-amide (XVII) is then converted to the aminoalkylamide (XVIII) by reaction with a secondary amine R⁴R³NH, typically at an elevated temperature in a solvent such as chloroform, dichloromethane or dimethylformamide, or more typically acetonitrile in the presence of an alkali metal carbonate base such as potassium carbonate. The aminoalkylamide (XVIII) is then deprotected (e.g. in the case of boc by treatment with an acid) and the amine reacted with an azide of the formula (XII) as described above to give a compound of the formula (X).

Compounds of the formula (XI) wherein G is CONH can be prepared by the sequence of reactions in Scheme 2.

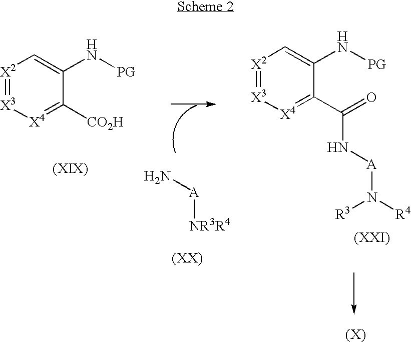

In Scheme 2, a protected ortho-aminoaryl- or heteroaryl carboxylic acid such as anthranilic acid is coupled with the alkylene diamine (XX) under amide forming conditions of the type described above. The amide (XXI) is then deprotected and reacted with an azide of the formula (XII) to give a compound of the formula (X).

Once formed, one compound of the formula (I), or a protected derivative thereof, can be converted into another compound of the formula (I) by methods well known to the skilled person. Examples of synthetic procedures for converting one functional group into another functional group are set out in standard texts such as Advanced Organic Chemistry, by Jerry March, 4^{th} edition, 119, Wiley Interscience, New York; Fiesers' Reagents for Organic Synthesis, Volumes 1-17, John Wiley, edited by Mary Fieser (ISBN: 0-471-58283-2); and Organic Syntheses, Volumes 1-8, John Wiley, edited by Jeremiah P. Freeman (ISBN: 0-471-31192-8)).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

Compounds made by the foregoing methods may be isolated and purified by any of a variety of methods well known to those skilled in the art and examples of such methods include recrystallisation and chromatographic techniques such as column chromatography (e.g. flash chromatography) and HPLC.

### Pharmaceutical Formulations

While it is possible for the active compound to be administered alone, it is preferable to present it as a pharmaceutical composition (e.g. formulation) comprising at least one active compound of the invention together with a pharmaceutically acceptable carrier, and optionally one or more additional excipients.

Accordingly, in another aspect, the invention provides a pharmaceutical composition comprising a compound of the formula (I) and a pharmaceutically acceptable carrier.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)-aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediaminetetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

### Methods of Treatment

It is envisaged that the compounds of the formula (I) and sub-groups as defined herein will be useful in combination therapy with chemotherapeutic agents or radiation therapy in the prophylaxis or treatment of a range of proliferative disease states or conditions. Examples of such disease states and conditions are set out above.

Particular examples of chemotherapeutic agents that may be co-administered with the compounds of formula (I) include:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Hypoxia triggered DNA damaging agents (e.g. Tirapazamine)

The compounds may be administered over a prolonged term to maintain beneficial therapeutic effects or may be administered for a short period only. Alternatively they may be administered in a pulsatile or continuous manner.

The compounds of the invention will be administered in an effective amount, i.e. an amount which is effective to bring about the desired therapeutic effect. For example, the “effective amount” can be a quantity of compound which, when administered together with a chemotherapeutic agent to a subject suffering from cancer, slows tumour growth, ameliorates the symptoms of the disease and/or increases longevity. More particularly, when used in combination with radiation therapy, with a DNA-damaging drug or other anti-cancer drug, an effective amount of the Chk-1 inhibitor of the invention is the quantity in which a greater response is achieved when the Chk-1 inhibitor is co-administered with the DNA damaging anti-cancer drug and/or radiation therapy compared with when the DNA damaging anti-cancer drug and/or radiation therapy is administered alone. When used as a combination therapy, an “effective amount” of the DNA damaging drug and/or an “effective” radiation dose are administered to the subject, which is a quantity in which anti-cancer effects are normally achieved. The Chk-1 inhibitors of the invention and the DNA damaging anti-cancer drug can be co-administered to the subject as part of the same pharmaceutical composition or, alternatively, as separate pharmaceutical compositions. When administered as separate pharmaceutical compositions, the Chk-1 inhibitor of the invention and the DNA-damaging anti-cancer drug (and/or radiation therapy) can be administered simultaneously or at different times, provided that the enhancing effect of the Chk-1 inhibitor is retained.

The amount of Chk-1 inhibitor compound of the invention, and the DNA damaging anti-cancer drug and radiation dose administered to the subject will depend on the type and severity of the disease or condition and on the characteristics of the subject, such as general health, age, sex, body weight and tolerance to drugs. The skilled person will be able to determine appropriate dosages depending on these and other factors. Effective dosages for commonly used anti-cancer drugs and radiation therapy are well known to the skilled person.

The compounds are generally administered to a subject in need of such administration, for example a human or animal patient, preferably a human.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound can be administered on a daily basis or on a repeat basis every 2, or 3, or 4, or 5, or 6, or 7, or 10 or 14, or 21, or 28 days for example.

In one particular dosing schedule, a patient will be given an infusion of a compound for periods of one hour daily for up to ten days in particular up to five days for one week, and the treatment repeated at a desired interval such as two to four weeks, in particular every three weeks.

More particularly, a patient may be given an infusion of a compound for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

### EXAMPLES

The invention will now be illustrated, but not limited, by reference to the specific embodiments described in the following examples.

In the examples, the following abbreviations are used.

DCM dichloromethane
DMSO dimethylsulphoxide
EtOAc ethyl acetate
IPA isopropyl alcohol
MeOH methanol
NMR nuclear magnetic resonance
RT room temperature
SiO₂ silica
TEA triethylamine
THF tetrahydrofuran

Proton magnetic resonance (¹H NMR) spectra were recorded on a Bruker 400 instrument operating at 400 MHz, in DMSO-d₆ or MeOH-d₄ (as indicated) at 27° C., unless otherwise stated and are reported as follows: chemical shift δ/ppm (number of protons, multiplicity where s=singlet, d=doublet, t=triplet, q=quartet, m=multiplet, br=broad). The residual protic solvent was used as the internal reference.

Liquid chromatography and mass spectroscopy analyses were carried out using the system and operating conditions set out below. Where atoms with different isotopes are present and a single mass quoted, the mass quoted for the compound is the monoisotopic mass (i.e. ³⁵Cl; ⁷⁹Br etc.)

### LC/TS Conditions

Samples were analysed by reverse phase HPLC-MS using a Waters 2795 Alliance HT HPLC, a Micromass ZQ mass spectrometer and a Waters 996 photodiode array UV detector. The LC-MS used electrospray ionisation and one of two different chromatography systems, as follows.

### Solvents

C=1.58 g ammonium formate in 2.5 L water+2.5 mL Ammonia solution
D=2.5 L Acetonitrile+132 mL (5%) solvent C+2.5 mL Ammonia solution

Chromatography

| | |
|---|---|
| Column | Phenomenex Gemini C18, |
| | 5 um, 4.6 × 30 mm |
| Injection Volume | 5 μL |
| UV detection | 220 to 400 nm |
| Column Temperature | 35° C. |

| Time | A % | B % | C % | D % | Flow (mL/min) |
|---|---|---|---|---|---|
| 0.00 | 0.0 | 0.0 | 95.0 | 5.0 | 2.000 |
| 4.25 | 0.0 | 0.0 | 5.0 | 95.0 | 2.000 |
| 5.80 | 0.0 | 0.0 | 5.0 | 95.0 | 2.000 |
| 5.90 | 0.0 | 0.0 | 95.0 | 5.0 | 2.000 |
| 7.00 | 0.0 | 0.0 | 95.0 | 5.0 | 2.000 |

Mass Spectrometer

| Ionization mode: | Positive | | Negative | |
|---|---|---|---|---|
| Capillary Voltage: | 3.20 | kV | −3.00 | kV |
| Cone Voltage: | 30 | V | −30 | V |
| Source Temperature: | 110° | C. | 110° | C. |
| Desolvation Temperature: | 350° | C. | 350° | C. |
| Cone Gas Flow: | 30 | L/Hr | 30 | L/Hr |
| Desolvation Gas Flow: | 400 | L/Hr | 400 | L/Hr |
| Scan duration: | 0.50 | seconds | 0.50 | seconds |
| Interscan delay: | 0.20 | seconds | 0.20 | seconds |
| Mass range: | 80 to 1000 | AMU | 80 to 1000 | AMU |

### Example 1

### 1-[5-Chloro-2-(3-dimethyloxyamino-propoxy)-phenyl]-3-pyrazin-2-yl-urea

### 1A. 5-Chloro-2-(3-dimethylamino-propoxy)-phenylamine

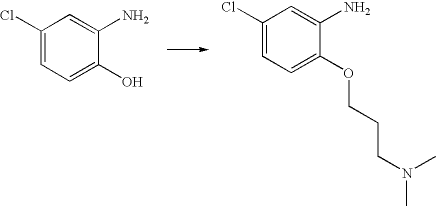

To a mixture of sodium methoxide (3.9 g) in IPA (75 ml) and toluene (75 ml) was added 2-amino 4-chlorophenol (5 g). (3-Chloro-propyl)-dimethyl-amine hydrochloride (5.5 g) was added and the reaction mixture was heated at reflux (100° C.) for 5 hours. On completion, the solvent was removed under reduced pressure and the resulting residue was extracted with ether (2×50 ml). The combined organic extracts were dried over sodium sulphate. The solvent was removed under reduced pressure to give a residue, which was further purified by column chromatography (SiO₂, 89:3:8 EtOAc:TEA:MeOH) to yield 4.2 g of the desired product (53%).

### 1B. Pyrazine-2-carbonyl azide

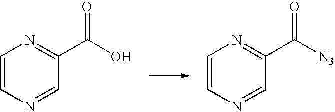

Diphenyl phosphorylazide (8.6 g) was added dropwise to a solution of pyrazine-2-carboxylic acid (3 g) in THF (30 ml) and TEA (9 ml) and the resulting reaction mixture was stirred at RT for 2 hours. On completion, THF was removed under reduced pressure to give a residue, which was further purified by column chromatography (SiO₂, gradient 10-40% EtOAc/n-hexane) yielding the desired product (2.5 g, 70%).

### 1C. 1-[5-Chloro-2-(3-dimethyloxyamino-propoxy)-phenyl]-3-pyrazin-2-yl-urea

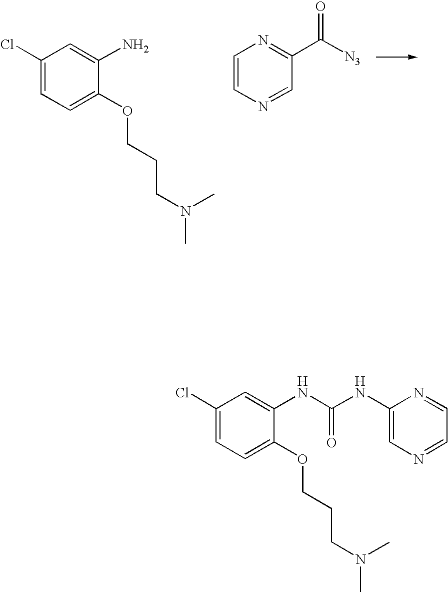

Pyrazine-2-carbonyl azide (2.5 g) and 5-chloro-2-(3-dimethylamino-propoxy)-phenylamine (7.1 g) in toluene (25 ml) were refluxed (110° C.) under a nitrogen atmosphere for 5 minutes. On completion, the reaction mixture was cooled to RT and the toluene removed under reduced pressure. The resulting residue was washed with diethyl ether to give the desired product (2.7 g, 30%).

N.M.R. (DMSO) δ 10.49 (s, 1H), 8.81 (br s, 1H), 8.38 (s, 2H), 8.35 (s, 1H), 7.13 (s, 1H), 4.19 (t, 2H), 2.63-2.58 (m, 2H), 2.3 (s, 6H), 2.12-2.06 (m, 2H)

LC/MS retention time: 2.90 E S⁺: 350

### 1D. N-Benzylidene-benzenesulphonamide

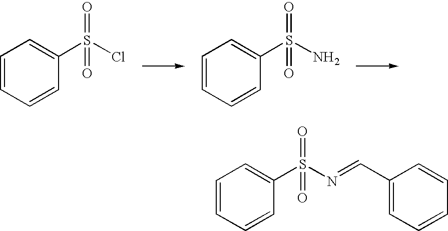

A solution of benzenesulphonyl chloride (10 g) in methanol (150 ml) was cooled to 0° C. and ammonia gas was bubbled into the reaction mixture over a period of 15 minutes. The reaction mixture was further stirred overnight at RT. On completion, the methanol was removed under reduced pressure and water (100 ml) was added. The reaction mixture was extracted with ethyl acetate (2×100 ml). The combined organic extracts were dried over sodium sulphate. The solvent was removed under reduced pressure to give 8.6 grams of benzenesulphonamide (96%). The benzenesulphonamide (2 g) was taken together with benzaldehyde (1.34 g), amberlyst resin (0.2 g) and molecular sieves (2 g) in dry toluene (20 ml) and refluxed (110° C.) for 30 minutes (till evolution of water ceases). The reaction mixture cooled to RT (without stirring) filtered through a Celite® bed and washed with toluene (40 ml). Finally, the toluene was removed under reduced pressure and the obtained a residue that when kept in the refrigerator for 30 minutes yielded a solid that was triturated with n-pentane (20 ml) filtered and dried for 15-20 min to give the desired product (2.5 g, 89%).

### 1E. 2-Benzenesulphonyl-3-phenyl-oxaziridine

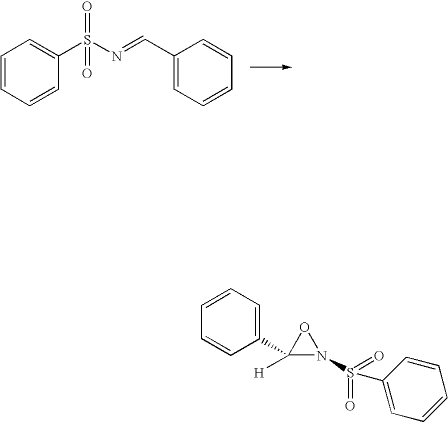

N-Benzylidene-benzenesulphonamide was taken together with a saturated solution of sodium bicarbonate (12.5 ml) and N-benzyl-N,N-diethylethanaminium chloride (0.25 g) and cooled to 0° C. 3-Chloroperbenzoic acid (3 g) in chloroform (22.5 ml) was added dropwise to the reaction mixture over a period of 15 min at 0° C. and stirring maintained for 1 hour. On completion, the organic layer was separated and washed with water (20 ml), 10% Na₂SO₃ solution (20 ml), sat. NaHCO₃ (20 ml) and sat. NaCl (20 ml). The organic layer was dried over potassium carbonate, filtered and the chloroform removed under reduced pressure (below 40° C.). The crude product was treated with n-Pentane (5 ml) and the resulting solid filtered. This solid was further triturated with ethyl acetate (14 ml) and then aged with n-Pentane (14 ml) overnight in the refrigerator. The resulting mixture was filtered and dried to yield the title compound (1.4 g, 48%).

### 1F. N-methyl-[3-[4-chloro-2-(pyrazin-3-yl-ureido)]-phenoxy]propylamine-N-Oxide

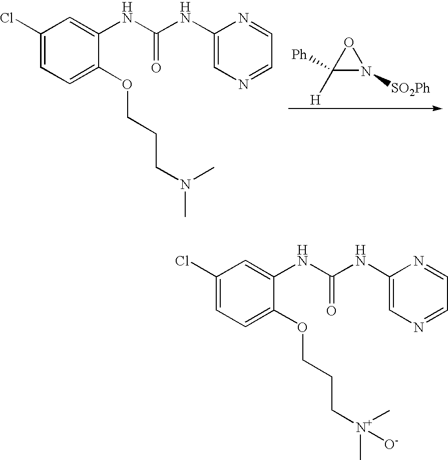

A solution of 2-benzenesulphonyl-3-phenyl-oxaziridine (0.24 g) in DCM (3 ml) was cooled to −20° C. and 1-[5-chloro-2-(3-dimethylamino-propoxy)-phenyl]-3-pyrazin-2-yl-urea (0.3 g) was added dropwise in DCM (6 ml). The reaction mixture was stirred for a further 30 min at −20° C. On completion, the DCM was removed under reduced pressure and the residue washed with acetone (3×3 ml) to yield the title compound.

N.M.R. (DMSO) δ 11.03 (br s, 1H), 10.85 (br s, 1H), 9.11 (s, 1H), 8.39 (s, 1H), 8.3-8.28 (m, 2H), 7.13 (d, 1H), 7.05 (dd, 1H), 4.22 (t, 2H), 3.66 (t, 2H), 3.32 (s, 6H), 2.37-2.31 (m, 2H) LC/MS rt 1.93 ES+ 366.

### Example 2

### 2A. (5-Methyl-pyrazin-2-yl)-carbamic acid phenyl ester

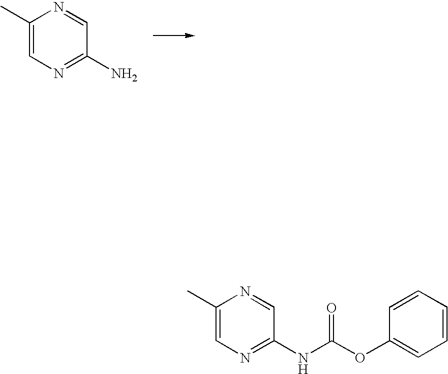

DMAP (5%) was added to a solution of 5-methyl-pyrazin-2-ylamine (10 mmoles) in pyridine (50 ml) which was further treated with phenyl chloroformate (1.2 eq) and the reaction stirred at room temperature overnight. The mixture was added to 100 g of ice and the precipitate was filtered and washed with water and dried to give the title compound.

### 2B. 1-[5-Chloro-2-(3-dimethylamino-propoxy)-phenyl]-3-(5-methyl-pyrazin-2-yl)-urea

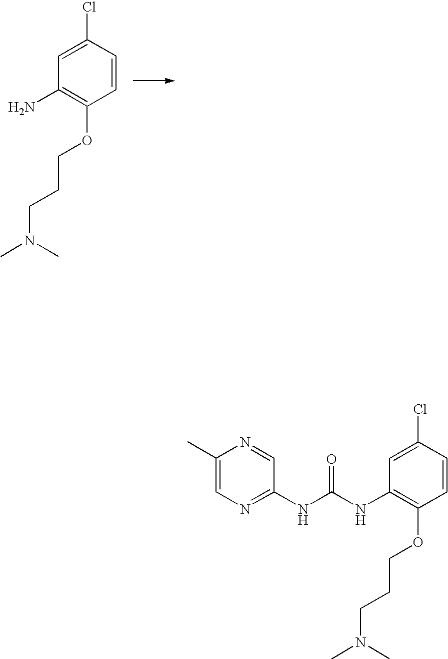

Compound 2A (1 mmole) was dissolved in dioxane (3 ml) and 5-chloro-2-(3-dimethylamino-propoxy)-phenylamine (1.0 mmole) was added. The mixture was heated to 120° C. for 15 minutes using microwave irradiation and then cooled to room temperature. The precipitate formed was filtered and washed with diethylether and dried to give the title compound.

### 2C. 1-[5-Chloro-2-(3-dimethyloxyamino-propoxy)-phenyl]-3-(5-methyl-pyrazin-2-yl)-urea

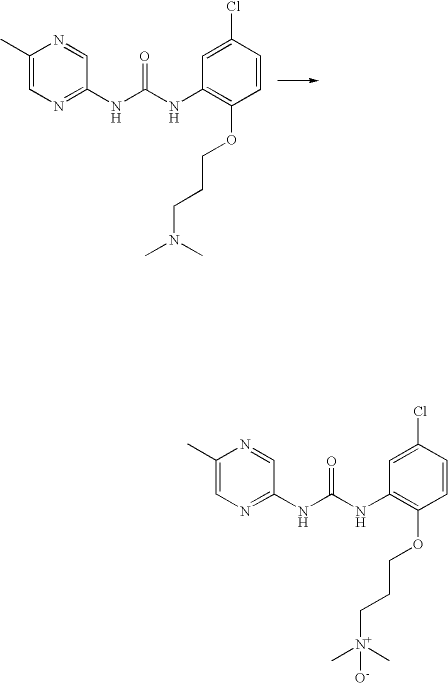

Compound 2B (100 mg) was suspended in DCM (5 ml) and treated at room temperature with 2-benzenesulfonyl-3-phenyl-oxaziridine (1.2 eq). The reaction mixture was stirred overnight, the precipitate filtered, washed (DCM and diethylether) and dried to give the title compound. N.M.R. (DMSO) δ 11.20 (br s, 1H), 11.05 (br s, 1H), 9.09 (br s, 1H), 8.24 (br s, 1H), 8.23 (d, 1H), 7.08 (d, 1H), 6.96 (dd, 1H), 4.14 (t, 2H), 3.55 (t, 2H), 3.22 (s, 6H), 2.41 (s, 3H), 2.26 (quint, 2H). LC/MS rt 1.92 ES+ 380.

### Example 3

### 1-[5-Chloro-2-(3-dimethyloxyamino-propoxy)-phenyl]-3-(5-cyano-pyrazin-2-yl)-urea

### 3A. (5-Cyano-pyrazin-2-yl)-carbamic acid phenyl ester

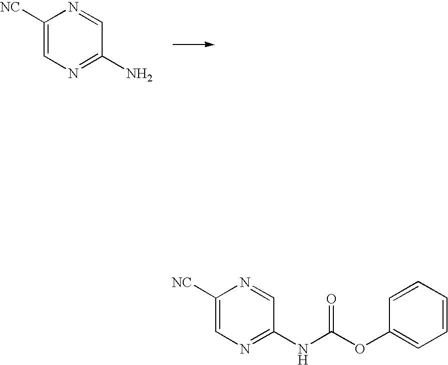

The title compound was prepared from 2-amino-5-cyanopyrazine and phenyl chloroformate following the protocol described in Example 2A.

### 3B. 1-[5-Chloro-2-(3-dimethylamino-propoxy)-phenyl]-3-(5-cyano-pyrazin-2-yl)-urea

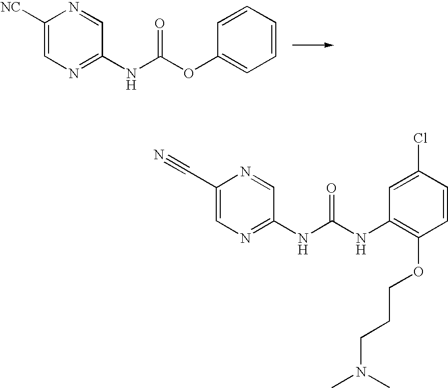

Compound 3A was reacted with 5-chloro-2-(3-dimethylamino-propoxy)-phenylamine following the protocol described in Example 2B to give the title compound.

### 3C. 1-[5-Chloro-2-(3-dimethyloxyamino-propoxy)-phenyl]-3-(5-cyano-pyrazin-2-yl)-urea

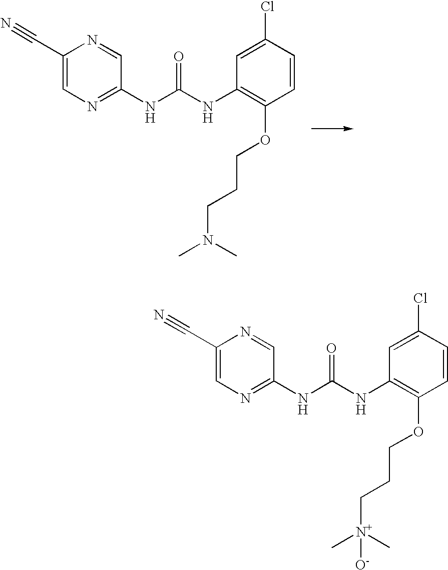

Compound 3B was subjected to N-oxidation with 2-benzenesulfonyl-3-phenyl-oxaziridine by following the protocol described in Example 2C to give the title compound. N.M.R. (DMSO) δ 11.31 (br s, 1H), 9.37 (d, 1H), 8.20 (d, 1H), 7.12 (d, 1H), 7.02 (dd, 1H), 4.15 (t, 2H), 3.58 (t, 2H), 3.24 (s, 6H), 2.29-2.11 (m, 2H). LC/MS rt 2.19 ES+ 391

### Example 4

### 1-[5-Chloro-2-(3-dimethyloxylamino-propoxy)-phenyl]-3-(5-cyano-pyridin-2-yl)-urea

### 4A. (5-Cyano-pyridin-2-yl)-carbamic acid phenyl ester

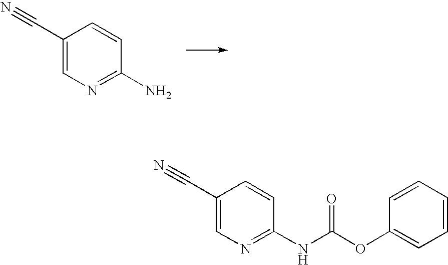

The title compound was prepared from 2-amino-5-cyanopyridine and phenyl chloroformate following the protocol described in Example 2A.

### 4B. 1-[5-Chloro-2-(3-dimethylamino-propoxy)-phenyl]-3-(5-cyano-pyridin-2-yl)-urea

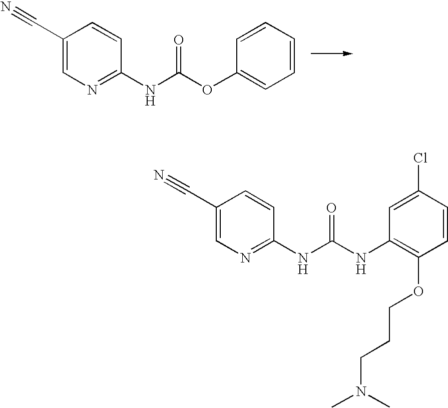

Compound 4A was reacted with 5-chloro-2-(3-dimethylamino-propoxy)-phenylamine following the protocol described in Example 2B to give the title compound.

### 4C. 1-[5-Chloro-2-(3-dimethyloxylamino-propoxy)-phenyl]-3-(5-cyano-pyridin-2-yl)-urea

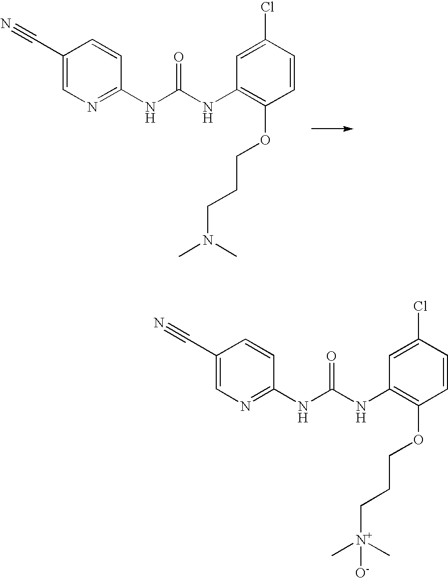

Compound 4B was subjected to N-oxidation with 2-benzenesulfonyl-3-phenyl-oxaziridine by following the protocol described in Example 2C to give the title compound. N.M.R. (DMSO) δ 11.32 (br s, 1H), 11.11 (br s, 1H), 8.74 (s, 1H), 8.21 (d, 1H), 8.15 (dd, 1H), 7.91 (br s, 1H), 7.08 (d, 1H), 7.00 (dd, 1H), 4.16 (t, 2H), 3.58 (t, 2H), 3.25 (s, 6H), 2.30-2.25 (m, 2H). LC/MS rt 2.14 ES+ 390

### Example 5

### 1-[5-Chloro-2-(1-methyl-1-oxy-piperidin-4-yloxy)-phenyl]-3-(5-cyano-pyrazin-2-yl)-urea

### 5A. 1-(5-Chloro-2-hydroxy-phenyl)-3-(5-cyano-pyrazin-2-yl)-urea

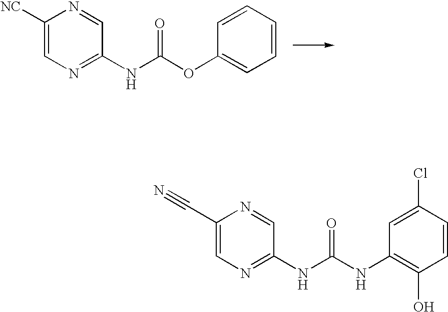

2-Amino-4-chloro-phenol (1.0 mmole) and (5-cyano-pyrazin-2-yl)-carbamic acid phenyl ester (1.0 mmole) were heated to 140° C. for 30 minutes using microwave irradiation in dioxane (4 ml). Once cooled to room temperature, the resulting precipitate was filtered and washed with diethylether and further dried to afford 270 mg of the title compound (93% yield).

5B. 1-[5-Chloro-2-(1-methyl-piperidin-4-yloxy)-phenyl]-3-(5-cyano-pyrazin-2-yl)-urea

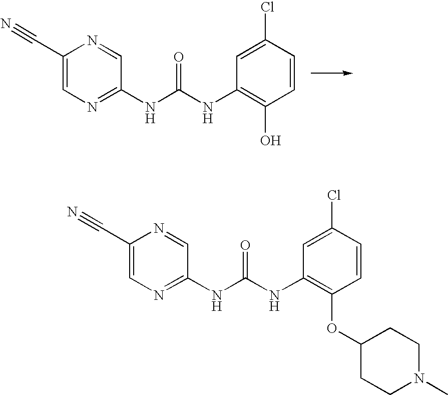

1-Methyl-piperidin-4-ol (0.55 mmole), triphenylphosphine (0.55 mmol), (0.55 mmole) DIAD and 5A (0.5 mmole) were stirred in of THF (3 ml) at room temperature overnight. The reaction mixture was then concentrated and further purified by column chromatography using a gradient of 0-10% saturated NH₃/MeOH and DCM to yield the title compound (31 mg, 16% yield).

5C. 1-[5-Chloro-2-(1-methyl-1-oxy-piperidin-4-yloxy)-phenyl]-3-(5-cyano-pyrazin-2-yl)-urea

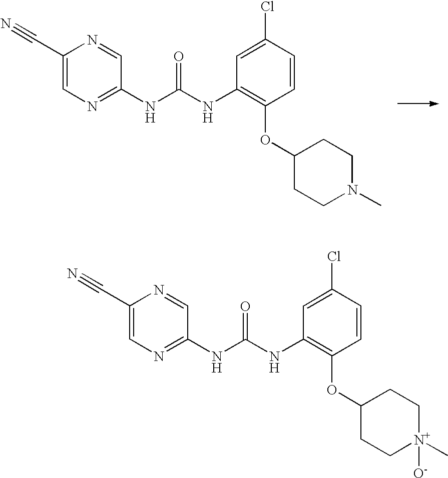

Compound 5B was subjected to N-oxidation following the protocol in Example 2C to give the title compound. N.M.R. (DMSO) δ 11.76 (br s, 1H), 10.23 (br s, 1H), 9.06 (br s, 1H), 8.93 (br s, 1H), 8.27 (s, 1H), 7.20 (br d, 1H), 7.09 (d, 1H), 4.58-4.48 (m, 1H), 3.53-3.11 (m, 6H), 3.15 (s, 3H), 2.02-1.91 (m, 2H) LC/MS rt 2.05 ES+ 403

### Biological Activity

### Example 6

### Chk-1 Kinase Inhibiting Activity

The N-oxide compounds of the invention and their tertiary amine precursors were tested for activity against the Chk-1 kinase.

The Chk-1 assay was conducted at Upstate Ltd, Gemini Crescent, Dundee Technology Park, Dundee, DD2 1 SW, UK in accordance with the protocol set out below. In a final reaction volume of 25 μl, CHK1 (h) (5-10 mU) is incubated with 8 mM MOPS pH 7.0, 0.2 mM EDTA, 200 μM KKKVSRSGLYRSPSMPENLNRPR, 10 mM MgAcetate and γ-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction mixture is then spotted onto a P30 filtermat and washed three times for 5 minutes in 75 mM phosphoric acid and once in methanol prior to drying and scintillation counting.

The IC₅₀ values for each of the compounds tested are set out in Table 3 below.

### Example 7

### hERG Binding Activity

The abilities of the N-oxide compounds of the invention and their tertiary amine precursors were tested using the assay described below.

Human recombinant HEK-293 cells stably transfected with a plasmid encoding the human potassium channel hERG were used to prepare membranes in modified HEPES pH 7.4 buffer using standard techniques. A 10 μg aliquot of membrane was incubated with 1.5 nM [³H] astemizole in the presence of varying concentrations of test compound for 60 minutes at 25° C. The membranes were then filtered and washed three times and the filters were counted to determine the [³H] astemizole specifically bound. From the counts, the extent of binding of the test compound and, in some cases, the IC₅₀ values of the test compounds, were determined.

The results are set out in Table 3 below.

### Example 8

### Functional hERG Assay

The N-oxide compounds of the invention and their tertiary amine precursors were each tested for hERG inhibiting activity using the method of Zhou Z, Gong Q, Ye B, Fan Z, Makielski J C, Robertson G A, January C T. Biophys J. 1998 January; 74(1):230-41.

Test compounds were tested in triplicate in a 5-point dose response curve on HEK-293 cells stably expressing the hERG potassium channel. Potassium current was measured using the patch clamp technique on a Molecular Devices Patch Express 7000. hERG channels were activated by 2 second pulses to +20 mV from a holding potential of −80 mV, and peak tail currents were recorded upon repolarization to −50 mV. This voltage-clamp pulse protocol was performed continuously during the experiment (vehicle control, test compound, washout, and positive control additions). An interpulse interval of 15 seconds allowed recovery from any residual inactivation. Test compounds were incubated with cells between 3-8 minutes until the current reached a steady state level, defined by a Standard Deviation of 0.01. After the final test compound concentration was tested, test compound was washed out with continuous perfusion of extracellular solution for 3 minutes, followed by application of positive control (10 μM Cisparide). If the positive control failed to achieve 100% inhibition the experiment was discarded.

Test compounds were diluted in 100% DMSO at 1000× the highest concentration to be tested, vortexed, and sonicated. Visual inspection determined that compounds were completely solubilized. Test compounds were then diluted into glass vials in 100% DMSO for all test concentrations at 1000× the final concentration to be tested and vortexed. Test compounds were then diluted 1:1000 into extracellular solution and vortexed to achieve final concentration for testing. Final DMSO concentration was 0.1% for all concentrations of test compounds, Vehicle (negative) control, and Cisapride (positive) control in extracellular solution. The compositions of the solutions used were as follows:

Intracellular Soln (mM): KCl 130, EDTA 5, MgCl₂ 5, HEPES10, Na-ATP 5, pH=7.2
Extracellular Soln (mM): NaCl 137, KCl 4, CaCl₂ 1.8, MgCl₂ 1, Dextrose 11, HEPES 10, pH=7.4

### Vehicle: 0.1% DMSO

The IC₅₀ values for each of the compounds are set out in Table 3 below.

TABLE 3

| | Chk1 | HERG Binding | HERG Functional |
|---|---|---|---|
| Example | IC₅₀ (μM) | IC₅₀ (μM) | IC₅₀ (μM) |
| 1C (tert. amine) | 0.041 | 5.030 | 0.108 |
| 1F (N-oxide) | 0.071 | 35% @ 100 uM | 26.700 |
| 2B (tert. amine) | 0.051 | 2.98 | 0.610 |
| 2C (N-oxide) | 0.128 | 3% @ 100 uM | >30.000 |
| 3B (tert. amine) | <0.010 | 5.12 | 0.435 |
| 3C (N-oxide) | 0.024 | 46% @ 100 uM | 16.319 |
| 4B (tert. amine) | 0.279 | 0.445 | 0.13 |
| 4C (N-oxide) | 0.581 | 7.67 | 2.44 |

The results shown in Table 3 illustrate that although N-oxide formation formation results in a slight reduction in activity against Chk-1, the N-oxides are nevertheless still potent inhibitors, each having activity in the sub-micromolar range. Importantly, however, the N-oxide compounds all have a much greater separation of Chk-1 activity and HERG activity than the precursor tertiary amines.

### Example 9

### Sensitisation of HT29 Cells to the DNA Damaging Effects of SN38

Many anti-cancer drugs achieve their anti-cancer effects by causing DNA damage but resistance to such drugs can be a significant problem. One mechanism responsible for drug resistance is the prevention of cell cycle progression through the control of critical activation of a checkpoint pathway which arrests the cell cycle to provide time for repair, and induces the transcription of genes to facilitate repair, thereby avoiding immediate cell death. Chk-1 kinase is involved in controlling checkpoint arrests and, by inhibiting the activity of Chk-1, it should be possible to prevent checkpoint arrests, thereby enhancing the action of DNA damaging agents by allowing mitosis to occur before DNA repair is complete.

Studies were carried out to test the sensitising effect of compounds of the invention on the ability of the DNA damaging compound 7-ethyl-10-hydroxycamptothecin (SN38), an active metabolite of the anti-cancer drug irinotecan, to inhibit cell growth in HT29 cells.

Inhibition of cell growth was measured using the Alamar Blue assay, based on the ability of viable cells to reduce resazurin to its fluorescent product resorufin. Cell lines can be from the ECACC (European Collection of cell Cultures).

The effect of a compound of the invention (compound X) in combination with compound Y (SN38) was assessed using the following technique:

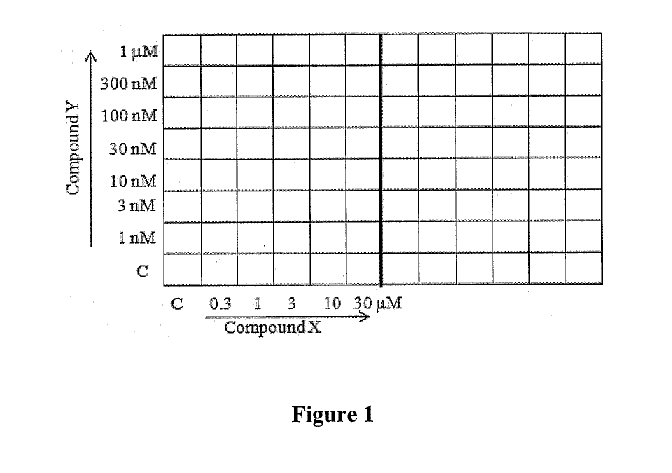

HT29 human cell lines were seeded onto 96-well plates at a concentration of 3000 cells per well and allowed to adhere for 16-24 hours prior to addition of compound or vehicle control (C) as shown in FIG. 1.

Compounds were added concurrently for 72 hours, following which 10% (v/v) Alamar Blue was added and incubated for a further 6 hours prior to determination of fluorescent product at 535 nM excitation/590 emission.

The IC₅₀ for Compound Y (SN38) in the presence of varying doses of Compound X was determined. Synergy was adjudged to have occurred when the IC₅₀ shifted down in the presence of sub-effective doses of Compound X. Additivity was considered to exist when the response to Compound Y and Compound X together resulted in an effect equivalent to the sum of the two compounds individually. Antagonistic effects were defined as those causing the IC₅₀ to shift upwards where the response of the two compounds was less than the sum of the effect of the two compounds individually.

The results are shown in Table 4 below.

TABLE 4

| Compound | | | |
|---|---|---|---|
| (Example No. | Concentration (μM) | SN38 IC₅₀ | fold shift |
| Ex. 2C | 0 | 52.60 | 1.0 |
| | 0.3 | 49.00 | 1.1 |
| | 1 | 34.00 | 1.5 |
| | 3 | 24.00 | 2.2 |
| | 10 | 16.50 | 3.2 |
| | 30 | 6.85 | 7.7 |
| Ex. 3C | 0 | 55.59 | 1.0 |
| | 0.3 | 13.57 | 4.1 |
| | 1 | 7.90 | 7.0 |
| | 3 | 3.90 | 14.3 |
| | 10 | 2.00 | 27.8 |
| Ex. 4C | 0 | 53.98 | 1.0 |
| | 0.3 | 33.48 | 1.6 |
| | 1 | 21.59 | 2.5 |
| | 3 | 10.90 | 5.0 |
| | 10 | 5.70 | 9.5 |
| | 30 | 3.59 | 15.0 |
| Ex. 1F | 0 | 43.81 | 1.0 |
| | 1.25 | 17.95 | 2.4 |
| | 2.5 | 10.04 | 4.4 |
| | 5 | 8.02 | 5.5 |
| | 10 | 5.09 | 8.6 |
| | 20 | 2.1 | 20.9 |

Data is N = 2

The data in Table 4 show that the compounds of this invention can sensitise HT29 cell cultures to the DNA damage induced by the action of SN-38. This sensitisation is in line with a mechanism involving Chk1 inhibition leading to G2/M abrogation and sensitisation of p53 compromised cells. Although the sensitising action of the compounds of the invention has been exemplified using HT29 cells, it is not limited to this cell type and it is envisaged that other cell types (e.g. p53 compromised cell types) will also be sensitised to DNA damaging agents by the compounds of the invention. Examples of such cell types include Co1o205, SKMe128, H322, OvCar3, MDA MB231 & U373MG.

### Example 10

### Kinase Selectivity Screen

The compound of Example 1F was tested (Upstate) against a number of other kinases and the results are set out in Table 5 below.

TABLE 5

| Target | ATP(μM) | % inhibition @ 10 μM |
|---|---|---|
| Aurora-A(h) | 15 | 14 |
| CDK2/cyclinE(h) | 120 | 0 |
| CHK2(h) | 70 | 71 |
| cKit(h) | 200 | 0 |
| cSRC(h) | 200 | 11 |
| EGFR(h) | 10 | 0 |
| FGFR1(h) | 200 | 92 |
| Flt3(h) | 200 | 73 |
| Fms(h) | 200 | 12 |
| IGF-1R(h) | 200 | 36 |
| JNK1α1(h) | 45 | 0 |
| KDR(h) | 90 | 38 |
| MAPK2(h) | 155 | 0 |
| MEK1(h) | 10 | 0 |
| NEK2(h) | 120 | 0 |
| PDGFRβ(h) | 200 | 1 |
| Pim-1(h) | 90 | 0 |
| PKA(h) | 10 | 0 |
| PKCθ(h) | 15 | 37 |
| Tie2(h) | 200 | 11 |
| Yes(h) | 45 | 0 |
| ZAP-70(h) | 15 | 5 |

N = 2

The results show that the compound of Example 1F is largely inactive or only poorly active against most other kinases apart from CHK2, FGFR1 and Flt3.

### Pharmaceutical Formulations

### Example 11

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

### vi) Injectable Formulation Iv

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-43. (canceled)

44. A compound of the formula (I⁰): 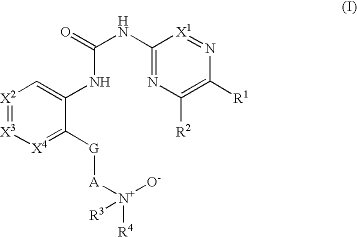
or a salt or tautomer thereof, wherein:
G is CH₂, O, NH, NHCO or CONH;
A is a group (CH₂)ₙ where n is 1 to 4 provided that when G is O or NH, n is at least 2;
X¹ is nitrogen or CH;
X² is nitrogen or a group CR⁵;
X³ is nitrogen or a group CR⁵;
X⁴ is nitrogen or CH; provided that no more than two of X², X³ and X⁴ are nitrogen;
R¹ is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups;
R² is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups; provided that no more than one of R¹ and R² can be an aryl or heteroaryl group;
or R¹ and R² together with the carbon atoms to which they are attached form a benzene ring;
R³ and R⁴ are the same or different and each is C₁₋₄ alkyl; or R³ and R⁴ together with the nitrogen atom to which they are attached form an azetidine, pyrrolidine, piperidine, piperazine, M-methylpiperazine or morpholine group; or R³ together with the nitrogen atom to which it is attached and the moiety A together form a saturated 5 to 7 membered heterocyclic ring optionally containing a second heteroatom ring member selected from O and S, wherein the heterocyclic ring is optionally substituted by 1 to 4 methyl groups, and R⁴ is C₁₋₄ alkyl;
R⁵ is hydrogen or a substituent R⁶;
R⁶ is halogen; hydroxy; trifluoromethyl; cyano; nitro; amino; mono- or di-C₁₋₄ hydrocarbylamino; a carbocyclic or heterocyclic group having from 3 to 12 ring members and optionally substituted by one or more substituents R⁷; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c} SO₂;
R^{b} is:
hydrogen;
a carbocyclic and heterocyclic group having from 3 to 12 ring members and being optionally substituted by one or more substituents R⁷;
a C₁₋₁₂ hydrocarbyl group optionally substituted by one or more substituents selected from hydroxy; oxo; halogen; cyano; nitro; carboxy; amino; mono- or di-C₁₋₈ non-aromatic hydrocarbylamino; and carbocyclic and heterocyclic groups having from 3 to 12 ring members optionally substituted by one or more substituents R⁷; wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c} is R^{b}, hydrogen or C₁₋₄ hydrocarbyl;
X¹ is O, S or NR^{c}; and
X² is ═O, ═S or ═NR^{c};
wherein R⁷ is selected from R⁶ provided that when the substituents R⁷ contain a carbocyclic or heterocyclic group having from 3 to 12 ring members, the said carbocyclic or heterocyclic group can be unsubstituted or substituted by one or more substituents R⁸; and
R⁸ is selected from R⁶ except that any carbocyclic or heterocyclic groups constituting or forming part of R⁸ may not bear a substituent containing or consisting of a carbocyclic or heterocyclic group but may optionally bear one or more substituents selected from halogen; hydroxy; trifluoromethyl; cyano; nitro; amino; mono- or di-C₁₋₄ hydrocarbylamino; or a group R^{a}—R^{bb}; where R^{a} is as hereinbefore defined and R^{bb} is hydrogen or a C₁₋₆ hydrocarbyl group optionally substituted by one or more substituents selected from hydroxy, oxo, halogen, cyano, nitro, carboxy, amino, mono- or di-C₁₋₄ saturated hydrocarbylamino and wherein one or more carbon atoms of the C₁₋₆ hydrocarbyl group may optionally be replaced by O, S, SO, SO₂, NR^{c}, X¹C(X²), C(X²)X¹ or X¹C(X²)X¹.

45. A compound according to claim 44 of the formula (Ia): 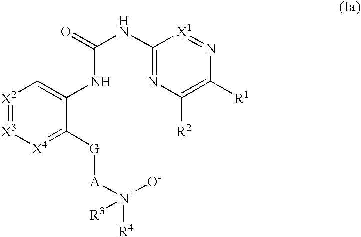
or a salt or tautomer thereof, wherein:
G is CH₂, O, NH, NHCO or CONH;
A is a group (CH₂)ₙ where n is 1 to 4 provided that when G is O or NH, n is at least 2;
X¹ is nitrogen or CH;
X² is nitrogen or a group CR⁵;
X³ is nitrogen or a group CR⁵;
X⁴ is nitrogen or CH; provided that no more than two of X², X³ and X⁴ are nitrogen;
R¹ is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups;
R² is hydrogen, cyano, C₁₋₄ alkyl, trifluoromethyl or a 5-6 membered monocyclic aryl or heteroaryl group containing up to 3 heteroatom ring members selected from O, N and S and being optionally substituted by one or two C₁₋₄ alkyl groups; provided that no more than one of R¹ and R² can be an aryl or heteroaryl group;
or R¹ and R² together with the carbon atoms to which they are attached form a benzene ring;
R³ and R⁴ are the same or different and each is C₁₋₄ alkyl; or R³ and R⁴ together with the nitrogen atom to which they are attached form an azetidine, pyrrolidine, piperidine, piperazine, M-methylpiperazine or morpholine group; and
R⁵ is hydrogen or a substituent R⁶;
R⁶ is halogen; hydroxy; trifluoromethyl; cyano; nitro; amino; mono- or di-C₁₋₄ hydrocarbylamino; a carbocyclic or heterocyclic group having from 3 to 12 ring members and optionally substituted by one or more substituents R⁷; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c} SO₂;
R^{b} is:
hydrogen;
a carbocyclic and heterocyclic group having from 3 to 12 ring members and being optionally substituted by one or more substituents R⁷;
a C₁₋₁₂ hydrocarbyl group optionally substituted by one or more substituents selected from hydroxy; oxo; halogen; cyano; nitro; carboxy; amino; mono- or di-C₁₋₈ non-aromatic hydrocarbylamino; and carbocyclic and heterocyclic groups having from 3 to 12 ring members optionally substituted by one or more substituents R⁷; wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c} is R^{b}, hydrogen or C₁₋₄ hydrocarbyl;
X¹ is O, S or NR^{c}; and
X² is ═O, ═S or ═NR^{c};
wherein R⁷ is selected from R⁶ provided that when the substituents R⁷ contain a carbocyclic or heterocyclic group having from 3 to 12 ring members, the said carbocyclic or heterocyclic group can be unsubstituted or substituted by one or more substituents R⁸; and
R⁸ is selected from R⁶ except that any carbocyclic or heterocyclic groups constituting or forming part of R⁸ may not bear a substituent containing or consisting of a carbocyclic or heterocyclic group but may optionally bear one or more substituents selected from halogen; hydroxy; trifluoromethyl; cyano; nitro; amino; mono- or di-C₁₋₄ hydrocarbylamino; or a group R^{a}—R^{bb}; where R^{a} is as hereinbefore defined and R^{bb} is hydrogen or a C₁₋₆ hydrocarbyl group optionally substituted by one or more substituents selected from hydroxy, oxo, halogen, cyano, nitro, carboxy, amino, mono- or di-C₁₋₄ saturated hydrocarbylamino and wherein one or more carbon atoms of the C₁₋₆ hydrocarbyl group may optionally be replaced by O, S, SO, SO₂, NR^{c}, X¹C(X²), C(X²)X¹ or X¹C(X²)X¹.

46. A compound according to claim 44, or a salt or tautomer thereof, wherein X¹ is CH.

47. A compound according to claim 44, or a salt or tautomer thereof, wherein R¹ is hydrogen or a substituent selected from cyano, C₁₋₃ alkyl, trifluoromethyl, six membered rings containing one or two nitrogen ring members and five membered rings containing a nitrogen ring member and optionally one or two further heteroatom ring members selected from nitrogen, oxygen and sulphur, wherein the six membered rings and five membered rings are each optionally substituted by one or two C₁₋₄ alkyl groups.

48. A compound according to claim 47, or a salt or tautomer thereof, wherein R¹ is selected from hydrogen, methyl, trifluoromethyl, cyano, pyridyl, oxazolyl and methyl-substituted triazolyl.

49. A compound according to claim 44, or a salt or tautomer thereof, wherein R² is hydrogen or a substituent selected from cyano, C₁₋₃ alkyl, trifluoromethyl, six membered rings containing one or two nitrogen ring members (preferably one nitrogen ring member) and five membered rings containing a nitrogen ring member and optionally one or two further heteroatom ring members selected from nitrogen, oxygen and sulphur, wherein the six membered rings and five membered rings are each optionally substituted by one or two C₁₋₄ alkyl groups.

50. A compound according to claim 49, or a salt or tautomer thereof, wherein R² is hydrogen.

51. A compound according to claim 44, or a salt or tautomer thereof, wherein the moiety: 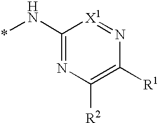
is selected from moieties A1 to A9: 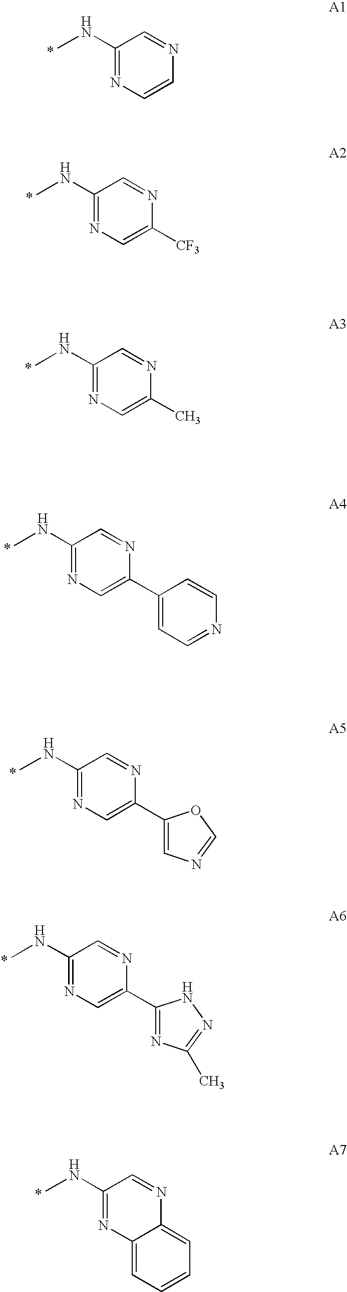 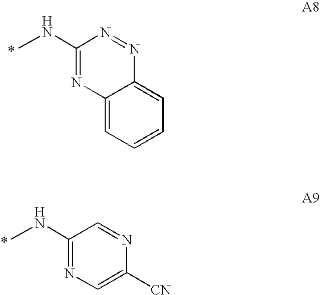
wherein the asterisk indicates the attachment of the moiety to the carbonyl group of the urea in formula (I).

52. A compound according to claim 44, or a salt or tautomer thereof, wherein G is O.

53. A compound according to claim 44, or a salt or tautomer thereof, wherein A is a group (CH₂)ₙ where n is 2 or 3.

54. A compound according to claim 44, or a salt or tautomer thereof, wherein R³ and R⁴ are the same or different and both are C₁₋₄ alkyl.

55. A compound according to claim 44, or a salt or tautomer thereof, wherein R³ together with the nitrogen atom to which it is attached and the moiety A together form a saturated 5 to 7 membered heterocyclic ring optionally containing a second heteroatom ring member selected from O and S, wherein the heterocyclic ring is optionally substituted by 1 to 4 methyl groups.

56. A compound according to claim 44, or a salt or tautomer thereof, wherein X² is CH or a group CR^{6a}, where R^{6a} is:
halogen;
hydroxy;
cyano;
nitro;
amino; mono- or di-C₁₋₄ alkylamino;
C₁₋₄ alkyl optionally substituted by one or more fluorine atoms, hydroxy, C₁₋₂ alkoxy, cyano, amino or mono- or di-C₁₋₄ alkylamino;
C₁₋₄ alkoxy optionally substituted by one or more fluorine atoms or C₁₋₂ alkoxy;
2-hydroxyethoxy; or
2-aminoethoxy;
and X³ is CH or a group CR⁶.

57. A compound according to claim 44, or a salt or tautomer thereof, wherein X³ is CH or CR^{6b} where R^{6b} is halogen; hydroxy; trifluoromethyl; cyano; amino; mono- or di-C₁₋₄ hydrocarbylamino; a carbocyclic group of 3 to 6 ring members or a heterocyclic group of 5 to 6 ring members, the carbocyclic and heterocyclic groups being optionally substituted by one or more substituents R^{7a}; or a group R^{a}—R^{b};
R^{a} is a bond, O, CO, X¹C(X²), C(X²)X¹, X¹C(X²)X¹, S, SO, SO₂, NR^{c}, SO₂NR^{c} or NR^{c} SO₂;
R^{b} is:
hydrogen;
a carbocyclic group of 3 to 6 ring members or a heterocyclic group of 5 to 6 ring members being optionally substituted by one or more substituents R^{7a};
a non-aromatic C₁₋₁₂ hydrocarbyl group optionally substituted by one or more substituents selected from hydroxy, oxo, halogen, cyano, carboxy, amino, mono- or di-C₁₋₈ non-aromatic hydrocarbylamino, a carbocyclic group of 3 to 6 ring members or a heterocyclic group of 5 to 6 ring members, the carbocyclic and heterocyclic groups being optionally substituted by one or more substituents R^{7a}; and wherein one or more carbon atoms of the C₁₋₁₂ hydrocarbyl group may optionally be replaced by O, S, SO, SO₂, NR^{c}, X¹C(X²), C(X²)X¹ or X¹C(X²)X¹;
R^{c}C is R^{b}, hydrogen or C₁₋₄ hydrocarbyl;
X¹ is O, S or NR^{c}; and
X² is ═O, ═S or ═NR^{c};
wherein R^{7a} is selected from R^{6b} provided that when the substituents R^{7a} contain a carbocyclic or heterocyclic group having from 3 to 12 ring members, the said carbocyclic or heterocyclic group can be unsubstituted or substituted by one or more substituents R^{8a}; and
R^{8a} is selected from R^{6b} except that any carbocyclic or heterocyclic groups constituting or forming part of R^{8a} may not bear a substituent containing or consisting of a carbocyclic or heterocyclic group but may optionally bear one or more substituents selected from
halogen;
hydroxy;
cyano;
nitro;
amino; mono- or di-C₁₋₄ alkylamino;
C₁₋₄ alkyl optionally substituted by one or more fluorine atoms, hydroxy, C₁₋₂ alkoxy, cyano, amino or mono- or di-C₁₋₄ alkylamino;
C₁₋₄ alkoxy optionally substituted by one or more fluorine atoms or C₁₋₂ alkoxy;
2-hydroxyethoxy; or
2-aminoethoxy.

58. A compound according to claim 44, or a salt or tautomer thereof, wherein the moiety: 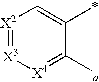
is selected from moieties B1 to B4: 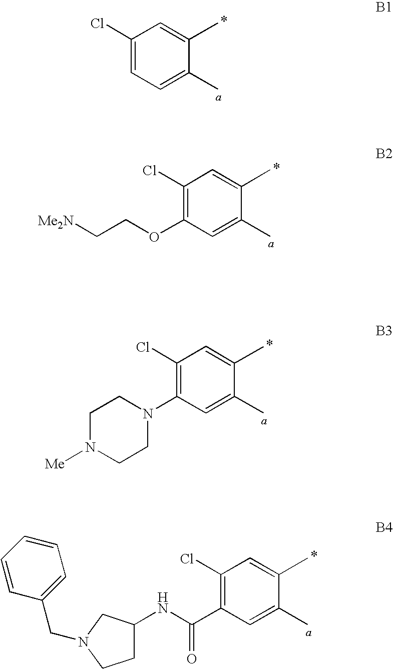
wherein the asterisk * denotes the point of attachment to the urea group in the compound of formula (I) and “a” denotes the point of attachment to the group G.

59. A compound according to claim 44 having the general formula (II) 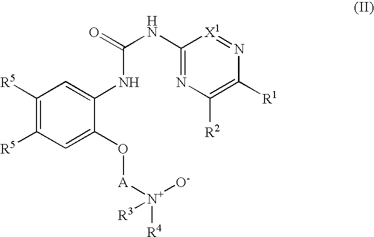
or a salt or tautomer thereof,
wherein X¹ is CH and R¹, R², R³, R⁴, R⁵, A and are as defined in claim 44.

60. A compound according to claim 44 which is selected from:
N-methyl-[3-[4-chloro-2-(pyrazin-3-yl-ureido)]-phenoxy]propylamine-N-oxide;
1-[5-chloro-2-(3-dimethyloxyamino-propoxy)-phenyl]-3-(5-methyl-pyrazin-2-yl)-urea;
1-[5-chloro-2-(3-dimethyloxyamino-propoxy)-phenyl]-3-(5-cyano-pyrazin-2-yl)-urea; and
1-[5-chloro-2-(1-methyl-1-oxy-piperidin-4-yloxy)-phenyl]-3-(5-cyano-pyrazin-2-yl)-urea.

61. A compound according to claim 44, or a salt or tautomer thereof, in the form of a pharmaceutical composition comprising the said compound or salt or tautomer thereof and a pharmaceutically acceptable carrier.

62. A method for the prophylaxis or treatment of a proliferative disease, which method comprises administering to a patient in combination with radiotherapy or chemotherapy a compound as defined in claim 44, or a salt or tautomer thereof.

63. A process for the preparation of a compound as defined in claim 44; which process comprises the reaction of a compound of formula (X): 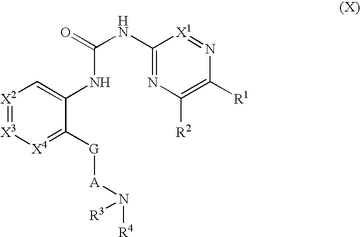
wherein X¹, X², X³, X⁴, G, A, R¹, R², R³, and R⁴ are as defined in claim 44, with a reagent capable of selectively oxidizing a non-aromatic amine to an N-oxide in the presence of a basic heteroaromatic nitrogen atom.
